(19) United States
(12) Patent Application Publication
(10) Pub. No.: US 2017/0188826 A1
(43) Pub. Date: Jul. 6, 2017

(54) APPARATUS FOR DETERMINING AND QUANTIFYING THE STAINING OF OCULAR STRUCTURES AND METHOD THEREFOR

(71) Applicant: Carl Zeiss Meditec AG, Jena (DE)

(72) Inventor: Mario Gerlach, Glienicke-Nordbahn (DE)

(21) Appl. No.: 14/986,212

(22) Filed: Dec. 31, 2015

(51) Int. Cl. A61B 3/14; A61B 3/13; A61B 3/12; A61B 3/00

(52) U.S. Cl. CPC A61B 3/14; A61B 3/0025; A61B 3/0033; A61B 3/13; A61B 3/12

(57) ABSTRACT

An apparatus has an image capture sensor which captures a reference image of a retro-illuminated unstained capsule of an eye. After the ocular structure has been stained with a dye for an ophthalmic surgical procedure a second image is captured as a measurement image via the image capture sensor. An evaluation module compares the reference image and the measurement image taken after the staining of the ocular structure. The evaluation module from the comparison of the reference and measurement images then determines the local light attenuation by the introduced dye. The staining of the eye can also be determined by only imaging a stained ocular structure of an eye and evaluating the images on the basis of predetermined acceptance values.

### FIELD OF THE INVENTION

This invention relates to the field of surgical ophthalmology, especially cataract surgery using colorants and laser methods on the capsular bag and on the natural eye lens. The invention further relates to the field of refractive corneal surgery for the application and control of corneal tattoos.

### BACKGROUND OF THE INVENTION

The application of dyes for staining and visualizing the anterior capsular surface in preparation for capsulorhexis has been established for years in cataract surgery. One of the most commonly used dyes for this clinical application is trypan blue. Trypan blue is very effective at dyeing the capsule as a result of its chemical binding characteristics and leads to a good visibility of the dyed structures due to its high absorption in the yellow to red spectral range.

Capsulorhexis is an example for an eye operation in the anterior of an eye. Here, a piece of the anterior capsular bag of an eye is scored in a circular region and opened, and the lens is removed through this hatch. The removed lens is replaced by an artificial lens or intraocular lens at the same position.

Aside from customary dyeing techniques for pure visualization of a manual rhexis, of combined methods using a dye and a laser for automatic preparation of the rhexis are currently being developed. In these methods, the dye and the laser are coordinated with one another in such a manner that the wavelength of the laser is effectively absorbed. The local optical energy input from the laser is transformed to heat through absorption and recombination. This resulting heat is used for the generation of a thermal cutting effect on the capsule through local coagulation and/or thermal degeneration of the capsule tissue.

Besides the primary thermal “cutting effect”, strong absorption of the laser light by the dye is also required for protection of the retina. Absorption by the dye ensures that the retinal irradiation values that occur lie below the maximum irradiation values permissible in each case. A sufficient quantity of the dye has to be situated and appropriately distributed in the interaction zone of the laser on the stained capsule. If the desired layer of dye has gaps or at least relatively weakly stained zones, then firstly the desired treatment effect is not achieved as a result of a lack of absorption and, secondly, the laser radiation impinges on the retina with virtually no attenuation and can irreparably damage the retina.

The laser therapy methods described above utilize the absorption resulting from the dye in order to protect the retina of the patient from damaging laser radiation. This only works if there is a sufficient amount of dye present in the interaction zone of the laser with the dyed capsule. If the desired dye layer contains gaps or at least less intensely dyed zones, then, due to the lacking absorption coefficient on the one hand, the desired treatment effect is not achieved and, on the other hand, the laser radiation hits the retina in a nearly undamped manner and can thus irreparably damage the retina.

What is disadvantageous in the current state of the art is that manual introduction of the dye and local resorption by the capsule are not comprehensively predictable processes. In this regard, it may be the case that in the desired treatment zone only an insufficient amount of dye is applied or the latter is distributed non-uniformly. Under certain circumstances, the surgeon might even totally fail to inject the dye.

To minimize risk, a known solution involves compelling the physician to work through corresponding check lists, visually inspecting the capsular staining using the surgical microscope, or else requesting corresponding manual confirmations of sufficient staining via treatment software.

### SUMMARY OF THE INVENTION

It is an object of the invention to provide an apparatus for measuring the distribution of a dye (especially local concentration and local spectral absorption) following the staining of ocular structures with the dye. It is a further object of the invention to provide a method for measuring the local distribution of a dye in an ocular structure stained with the dye.

The object is achieved in that a spatially resolved photometric measurement of the specific absorption of a test light beam through the dye resorbed in the tissue is carried out. The apparatus disclosed below makes use, in particular, of the spectral reflection characteristics of the ocular fundus as well as a double pass through the tissue to be analyzed.

With the aid of a surgical microscope the regions of the eye to be treated and/or analyzed are magnified for the physician. At least two functional beam paths in the surgical microscope are used therefor. A first beam path, an observation beam path, serves to magnify the image of the desired region. This observation beam path can, aside from the conventional viewing enablement, also contain further views or branches for cameras for display on image recording chips (CCD, CMOS, et cetera). The illumination beam path, which is connected to the viewing beam path and is guided (quasi) coaxially thereto, serves to directly illuminate the region to be shown in a low-reflective manner. In dependence upon the desired use, the illumination beam path can be adapted to the viewing situation through a switching in of diaphragms and/or optical filters. Additionally to the coaxial illumination, the surgical microscope can have a variety of different possibilities for environmental illumination.

Red reflection illumination or retro illumination is a particularly suitable illuminating method for cataract surgery. Here, the exit pupil of the light source is imaged in the working plane of the microscope according to the Koehler illumination method. After passing through the eye media, the light incident on the pupil of the eye impinges the retina where it is spectral specifically absorbed, scattered and reflected. The light reflected by the retina in a scattering manner illuminates the eye lens and the capsule from behind in a diffuse manner. The light source for this type of illumination, thus, virtually appears to be located behind the eye lens. For this reason, it is possible to achieve a high contrast transilluminate image of the transparent media (eye lens, capsule). If the incident illumination is done with white light, then the portion reflected by the retina is characterized by the spectral reflection characteristics of the retina. As the retina primarily reflects in the red spectral range, the returned reflection appears in red color (red reflex). Light with a shorter wavelength is for the most part absorbed by the retina. This is shown in FIG. 10 (from Delori, Burns: Fundus reflectance and the measurement of crystalline lens density, Vol. 13, No. 2 Feb. 1996 J. Opt. Soc. Am. A).

The object is achieved by an apparatus for measuring the distribution of a dye in an ocular structure. The apparatus includes: an image capture sensor configured to capture a reference image of an ocular structure of an eye in an unstained state; the image capture sensor being further configured to capture a measurement image of the ocular structure of the eye in a stained state; and, an evaluation module configured to determine the light attenuation between the reference image and the measurement image by comparing the reference image to the measurement image.

The object is further achieved by a further apparatus for measuring the distribution of a dye in an ocular structure. The apparatus includes: an image capture sensor configured to capture a first image of an ocular structure of an eye stained with a dye at a first wavelength and a second image of the ocular structure of the eye stained with the dye at a second wavelength; a data storage unit having a predetermined acceptance values of light attenuation stored thereon; and, an evaluation module configured to compare the determined light attenuation between the first image and the second image to the predetermined acceptance values.

The object is further achieved by a method for measuring the distribution of a dye in an ocular structure. The method includes the steps of: capturing a first image of the ocular structure in an unstained state; staining the ocular structure with a dye to attenuate light incident thereon; capturing a second image of the ocular structure in a stained state; and, determining the light attenuation by comparing the pixel intensities of the first image and the second image.

According to an embodiment of the invention, a reference image is initially taken via an image capture sensor. The ocular structure is then stained with a dye, for example trypan blue. After the ocular structure has been stained, the image capture sensor captures a measurement image of the stained ocular structure. The reference image and the measurement image are then compared to determine the local measurement light attenuation distribution, which corresponds to the local concentration or the local spectral absorption of a dye. The determined local distribution can then be compared to acceptance values which are predetermined values indicating whether it is safe to proceed with the application of the laser to the ocular structure in order to perform the surgical procedure.

### BRIEF DESCRIPTION OF THE DRAWINGS

The invention will now be described with reference to the drawings wherein:

FIG. 1 is a schematic showing a surgical microscope with the apparatus for determining and quantifying the staining of ocular structures;

FIG. 2 is a gray-scale representation of the local absorption with identification of critical locations and the region of interest;

FIG. 3 is a binary representation of the local absorption with identification of critical locations and the region of interest;

FIG. 4A shows the transmission of a dye, here trypan blue as an example, as a function of light wavelength;

FIG. 4B shows the reflection of light from the fundus of an eye as a function of light wavelength;

FIG. 5 shows the reflected and analyzable light with and without an application of trypan blue;

FIG. 6 shows capture and analysis in color channel 1 of a measurement image;

FIG. 7 shows capture and analysis in color channel 2 of the reference image;

FIG. 8 shows registration of the measurement image with respect to the reference image and the division of the measurement image by the reference image;

FIG. 9 shows the inversion of the division shown in FIG. 8; and,

FIG. 10 shows, juxtaposed, graphs of the fundus reflection as a function of wavelength and as a function of the age of a patient, respectively.

### DESCRIPTION OF THE PREFERRED EMBODIMENTS OF THE INVENTION

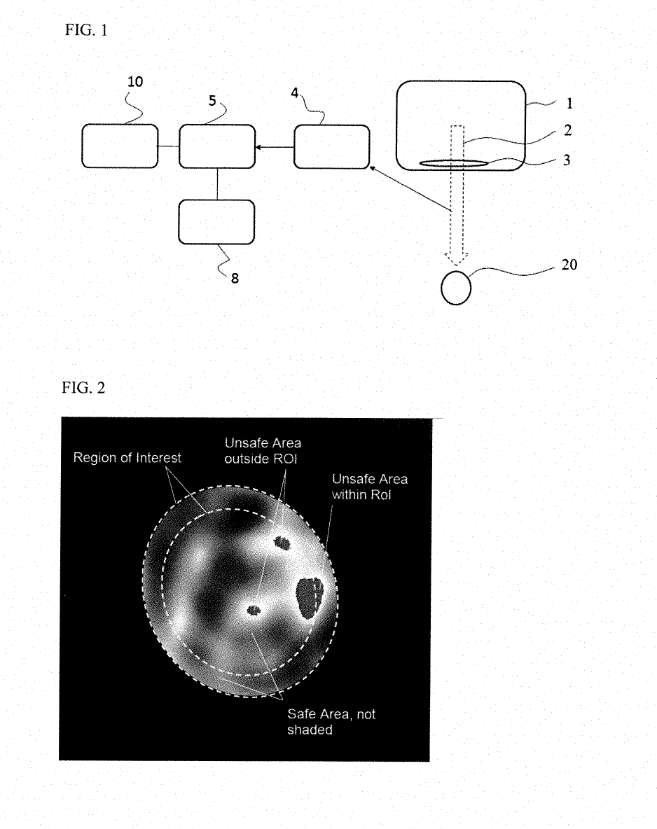

FIG. 1 shows a schematic illustration of a surgical microscope 1 with an apparatus for detecting and quantifying the staining of ocular structures according to the invention. The surgical microscope 1 includes a magnification unit 3, shown schematically. A line of sight 3 of a user through the surgical microscope 1 to a patient's eye 20 is depicted by the light path 2. The apparatus further includes an image capture sensor 4 for capturing images of an ocular structure and an evaluation module 5 configured to evaluate images taken by the image capture sensor 4 in accordance with a method of the invention.

In a first embodiment, the apparatus has an image capture sensor 4 which captures a reference image of a retro-illuminated unstained capsule. The reference image is preferably captured directly before staining in order to minimize any changes which may occur after the taking the reference image and prior to staining. After the ocular structure has been stained with a dye by, for example, a physician, a second image is captured as a measurement image via the image capture sensor.

The evaluation module 5 compares the pixel intensities of the reference image and of the measurement image taken after the staining of the ocular structure. By comparing the pixel intensities of the reference image and the measurement image, the evaluation module 5 then determines the local light attenuation by the introduced dye. Preferably, both images are automatically registered, aligned and, if appropriate, scaled with respect to one another before the evaluation by the evaluation module 5. The registering, aligning and scaling can be performed using known digital image referencing methods via feature comparison and/or cross-correlation. Furthermore, the evaluation of the local attenuation is preferably performed within a defined region of interest, for example whereat laser treatment is to be performed. It can also be provided that at least two adjacent pixels within the region of interest are used for averaging (metapixel) in the reference image as well as the measurement image. This can be used to reduce the disturbing influences caused by image noise. Besides the lateral averaging through adjacent pixels, it is also possible to use a multiplicity of sequential frames of the reference image as well as the measurement image for the averaging. These frames can each be registered with respect to each other prior to the averaging. In order to minimize the influence of fluctuations in the illumination intensity, the illumination intensity can, for example, either be measured or stabilized during the recording of the reference image and the measurement image. With the measured illumination intensity value, it is then possible to carry out normalization globally both in the reference image and in the measurement image. The reference and measurement images can, for example, be captured by the system camera of a surgical microscope or by a suitable supplementary camera.

The local attenuation of the light intensity determined in this manner can then be compared, preferably automatically, with previously determined acceptance values in a normative data base stored in a data storage unit 8 in order to make a decision as to whether the staining is sufficient at every location in the defined region of interest.

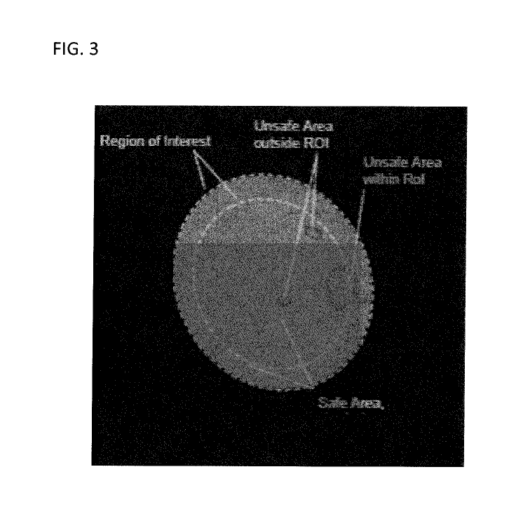

FIG. 2 is a gray-scale representation of the local absorption showing the region of interest and identifying the critical locations at which the dye distribution is inadequate and is thus unsafe for laser surgery. FIG. 1 also shows an input module 10 configured to enable a user to define a region of interest (ROI). FIG. 3 is a binary representation of the local absorption which identifies the region of interest and the critical locations. Whether critical locations are present in the region of interest can be performed by the physician or the evaluation module can determine the same automatically.

In a further embodiment, the evaluation can advantageously be expanded to evaluate not only a relative intensity comparison of pixel or metapixel values between the reference image and the measurement image, but to also evaluate the change in their respective color information (for example, RGB, CMYK, HSL et cetera). A “metapixel” is understood as a predefined aggregate (for example averaging 2×2 pixels to a single new metapixel) of single pixels after application of statistical process for example averaging to reduce noise. This additional information arises because the spectral absorption properties of the dye and the spectral reflection properties of the fundus differ. The evaluation of the color information thus makes it easier to distinguish the actual effect of the dye from illumination artifacts.

This embodiment can, for example, be realized in two different ways. A first option being an illumination with spectrally broadband light (white light) and image capture via a suitable color camera. A second option being temporary sequential illumination with quasi-monochromatic light with changing color function (for example, CIE XYZ, CIE RGB) or wavelength, for example Red→Green→Blue, or CIE-X→CIE-Y→CIE-Z or with a higher spectral discretion rate. Capture is then, for example, carried out via a monochromatic camera. For evaluation, the respective monochromatic frames then have to be linked with the light color function or wavelength used.

In a further embodiment, the apparatus is supplemented with a unit for obtaining a stable reference in the stained state. Capturing a reference image before staining can then be omitted or supplemented by the information of an in-process reference. The reference can be obtained from the interplay of the spectral absorption characteristics of the dye and the spectral reflection characteristics of the fundus. The relationships is described below on the basis of an example with trypan blue used as the dye.

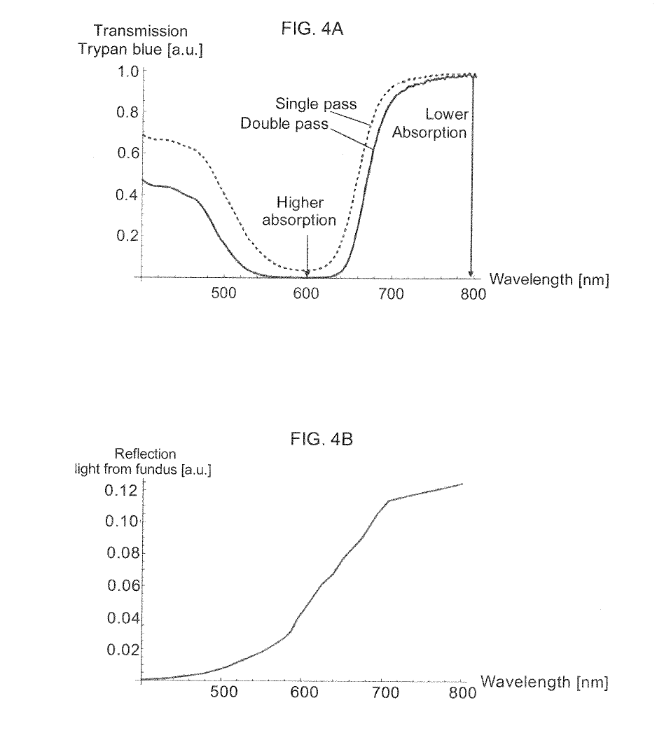

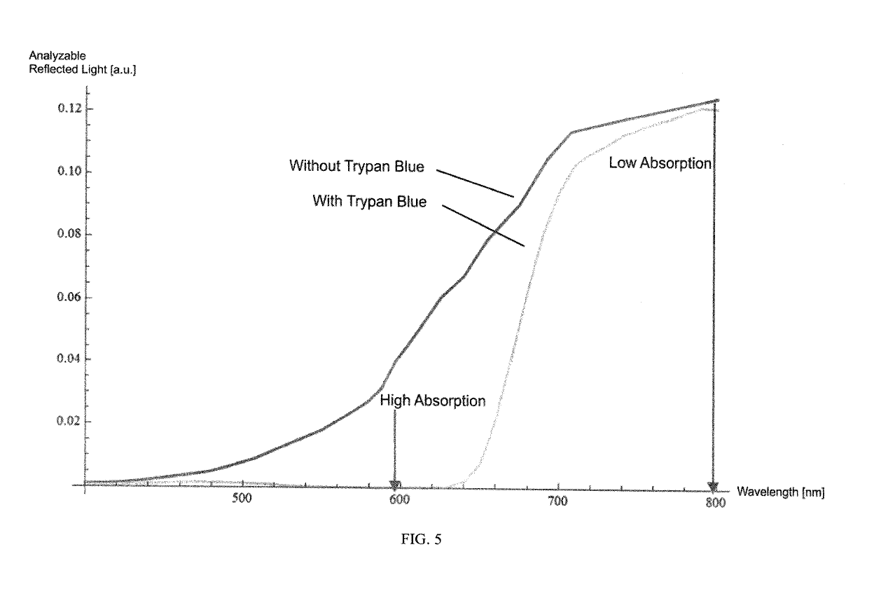

FIG. 4A shows the relationship between the wavelength of the light beam applied to the ocular structure and the transmission of the dye, trypan blue. The transmission for trypan blue is shown in single pass and double pass. FIG. 4A shows that the transmittance is clearly distinct at different wavelengths. The absorption exhibits a maximum at 600 nm, while the transmission in the broadband range is greatest. FIG. 4B shows the relationship of the wavelength of the light and the reflected light from the fundus. From FIG. 4B, it can be seen that the reflection factor increases with increasing wavelength.

When viewing FIGS. 4A and 4B together, it can be derived that a measurement of the absorption can preferably be performed at two different wavelengths at which, especially, the spectral absorptivity of the dye differs greatly. One wavelength serves as a process reference while the other wavelength is used as the measurement wavelength. Preferably a wavelength at which the transmittance is very high, especially close to 100%, is selected for the reference. A wavelength around 800 nm is particularly preferable. The measurement wavelength preferably lies close to the absorptivity maximum, for example around 600 nm. LEDs and/or a semiconductor laser are, for example, suitable light sources for these wavelengths.

FIG. 5 shows the reflected and analyzable light with and without an application of trypan blue.

From a spectrally separate but spatially resolved measurement of the attenuation of the fundus reflex of the measurement wavelength and the reference wavelength, a spatially resolved absorption measurement can be performed. Through the expansion of the system by a reference wavelength at very low absorption of the dye to be analyzed, a differentiation can be made as to whether an attenuation of the reflected light at the measurement wavelength actually occurs due to dye absorption or for example due to changing lighting conditions or local turbidity or cloudiness of the eye lens due to certain forms of cataracts. This differentiation and normalization possibilities enable the absorption through the dye to be evaluated more precisely.

In this embodiment, an image capture sensor captures a measurement image of a stained ocular structure at a first wavelength and a reference image of the stained ocular structure at a second wavelength. The first wavelength is selected such that the absorptivity of the dye is at or close to its maximum. The second wavelength is chosen such that the transmittance of the dye is high. The first and second images are registered with respect to each other and the evaluation module performs a division of the first image over the second image. The resulting quotient image is then compared to predetermined acceptance values stored in the data storage unit 8. The predetermined acceptance values stored on the data storage unit are derived from the interplay of the spectral absorption of the dye and the spectral reflection of the fundus. The comparison is then used to determine the local distribution of the dye and analyze whether the staining is sufficient to proceed with laser treatment. The results of the analysis can, for example, be presented in a similar manner as shown in FIGS. 2 and 3 and can also be used for controlling automatic safety interlocks. That is, as long as an insufficient absorption is present within the region of interest, the laser beam is blocked from being applied and the user can be notified with a corresponding message for addressing the problem.

The spectral separation can, for example, be achieved through chronologically alternating exclusive switching of the two light sources for the reference imaging process and the measurement imaging process. That is, the light sources for the reference image and the measurement image can be pulsed in an alternating manner. In this embodiment, the image of the camera for the image evaluation is tied to the corresponding wavelength. A color camera (system camera) as well as a monochrome camera can be used therefor. It may be necessary to perform a registration of the frames to each other. Alternatively, it is also possible to achieve a spectral separation with synchronized imaging via a corresponding dichroic beam splitter and camera associated with the corresponding wavelengths.

Both illuminating beam paths (reference and measurement) are guided together coaxially in the optical system and are configured in Kohler illuminating method for optimized fundus reflection.

As the method is to be performed on a living, movable eye, a change in illumination and/or reflection conditions can, for example, cause shadow casting with respect to the measurement beam. Such a reduction of the intensity as a result of shadowing would be interpreted by a non-continuous referencing method as an increased local dye concentration as a result of increased absorption. For this reason, the system according to the invention is, for example, configured as a continuously simultaneously referencing two-wavelength photometer.

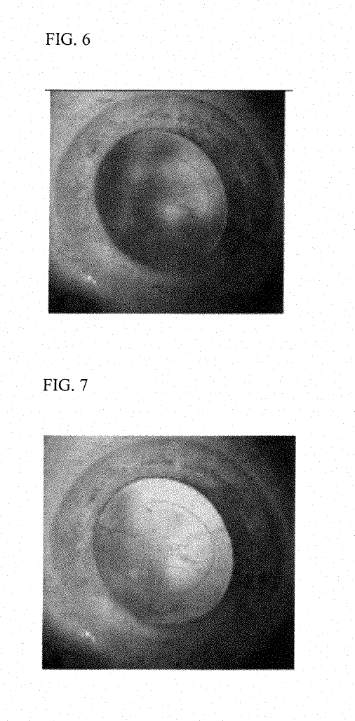

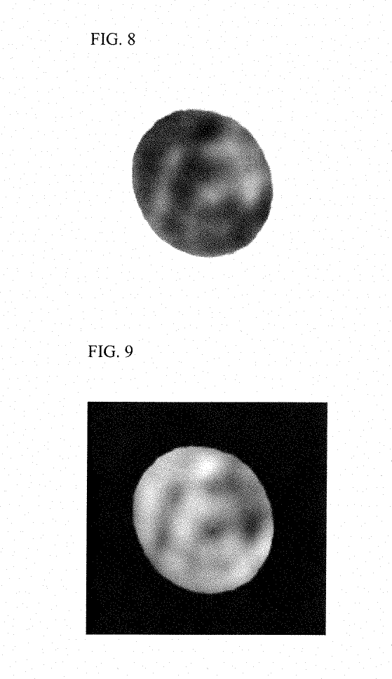

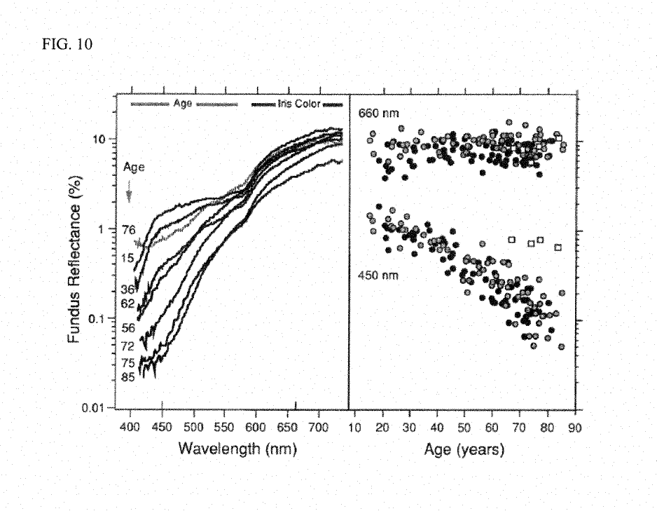

In an exemplary embodiment, a first image is captured and processed in a first color channel (FIG. 6). A second image is captured and processed in a second color channel (FIG. 7). The processing can for example include the steps of image separation into the referred color channels and noise suppression filtering. The measurement image is then registered with respect to the reference image and a division of the measurement image by the reference image is performed so as to generate a quotient image (FIG. 8). The quotient image is then preferably inverted (FIG. 9) and evaluated as to whether a sufficient quantity of dye is present. It is also possible to omit the inverting of the quotient image, however, this would then preferably be accounted for in the post-processing of the data. The evaluation as to whether a sufficient quantity of dye is present is performed with respect to predetermined acceptance values derived from the interplay of the spectral absorption of the dye and the spectral reflection of the fundus.

It is understood that the foregoing description is that of the preferred embodiments of the invention and that various changes and modifications may be made thereto without departing from the spirit and scope of the invention as defined in the appended claims.

What is claimed is:

1. An apparatus for measuring the local distribution of a dye in an ocular structure, the apparatus comprising:
an image capture sensor configured to capture a reference image of an ocular structure of an eye in an unstained state;
said image capture sensor being further configured to capture a measurement image of the ocular structure of the eye in a stained state; and,
an evaluation module configured to determine the light attenuation between said reference image and said measurement image by comparing said reference image to said measurement image.

2. The apparatus of claim 1, wherein:
said reference image has first pixel intensities;
said measurement image has second pixel intensities; and,
said evaluation module is configured to determine the light attenuation between said reference image and said measurement image by comparing said first pixel intensities to said second pixel intensities.

3. The apparatus of claim 1 further comprising:
a data storage unit connected to said evaluation unit and having a predetermined acceptance values of light attenuation stored thereon; and,
said evaluation module being further configured to compare the determined light attenuation between said reference image and said measurement image to said predetermined acceptance values.

4. The apparatus of claim 3 further comprising:
an input module configured to enable a user to define a region of interest; and,
said evaluation module being further configured to determine whether the light attenuation is below said predetermined acceptance at any location within said region of interest.

5. The apparatus of claim 1 further comprising:
an input module configured to enable a user to define a region of interest; and,
said evaluation module being further configured to determine the light attenuation within said region of interest between said reference image and said measurement image by comparing the same within said region of interest.

6. The apparatus of claim 1, wherein the apparatus is a surgical microscope.

7. The apparatus of claim 1, wherein the apparatus is a component of a surgical microscope.

8. An apparatus for measuring the local distribution of a dye in an ocular structure, the apparatus comprising:
an image capture sensor configured to capture a first image of an ocular structure of an eye stained with a dye at a first wavelength and a second image of said ocular structure of said eye stained with said dye at a second wavelength;
a data storage unit having a predetermined acceptance values of light attenuation stored thereon; and,
an evaluation module configured to compare the determined light attenuation between said first image and said second image to said predetermined acceptance values.

9. The apparatus of claim 8, wherein said first image is captured in a first color channel and said second image is captured in a second color channel.

10. The apparatus of claim 8, wherein said predetermined acceptance values are determined from a first characteristic for the transmission of said dye as a function of light wavelength and a second characteristic for the reflection of light from the fundus of an eye as a function of light wavelength.

11. The apparatus of claim 10 further comprising:
an input module configured to enable a user to define a region of interest whereat laser surgery is to occur; and,
said evaluation module being further configured to determine whether the light attenuation lies below said predetermined acceptance at any location within said region of interest.

12. The apparatus of claim 8 further comprising:
an input module configured to enable a user to define a region of interest; and,
said evaluation module being further configured to determine the light attenuation within said region of interest between said first image and said second image by comparing the same within said region of interest.

13. The apparatus of claim 8, wherein the apparatus is a surgical microscope.

14. The apparatus of claim 8, wherein the apparatus is a component of a surgical microscope.

15. A method for measuring the local distribution of a dye in an ocular structure, the method comprising the steps of:
capturing a first image of the ocular structure in an unstained state;
staining the ocular structure with the dye to attenuate light incident on said ocular structure;
capturing a second image of the ocular structure in a stained state; and,
determining the light attenuation by comparing the pixel intensities of the first image and the second image.

16. The method of claim 15, wherein said capturing of said reference image is performed directly prior to said staining of the ocular structure.

17. The method of claim 15 further comprising the step of comparing the determined light attenuation to predetermined acceptance values.

18. The method of claim 15, wherein said determining the light attenuation is performed on a defined region of interest of the ocular structure.

19. The method of claim 18 further comprising the step of comparing the determined light attenuation to predetermined acceptance values so as to determine whether staining is sufficient at every location in the region of interest.

20. The method of claim 15, wherein the ocular structure is a capsular bag.

21. The method of claim 15 further comprising the step of normalizing the first and second images.

22. A method for measuring the local distribution of a dye in an ocular structure comprising the steps of:
capturing a first image of the ocular structure in a stained state at a first wavelength;
capturing a second image of the ocular structure in the stained state at a second wavelength;
determining the light attenuation by comparing the pixel intensities of the first image and the second image to each other; and,
comparing the determined light attenuation to predetermined acceptance values stored on a data storage unit.

23. The method of claim 22, wherein said first image is captured in a first color channel and said second image is captured in a second color channel.
